(19) United States
(12) Patent Application Publication
(10) Pub. No.: US 2026/0216439 A1
(43) Pub. Date: Jul. 30, 2026

(54) MEDICAMENT DELIVERY DEVICE

(71) Applicant: SHL Medical AG, Zug (CH)

(72) Inventor: Joakim Gunnar Lindholm, Nacka Strand (SE)

(21) Appl. No.: 19/148,130

(22) PCT Filed: Dec. 13, 2023
(86) PCT No.: PCT/EP2023/085569

§ 371 Date: Jul. 15, 2025

(30) Foreign Application Priority Data
Jan. 24, 2023 (EP) 23152977.7

(51) Int. Cl. A61M 5/315; A61M 5/32

(52) U.S. Cl. CPC A61M 5/31501; A61M 5/3158; A61M 5/3202; A61M 2005/31508

(57) ABSTRACT

The present disclosure relates to a medicament delivery device comprising a housing having a longitudinal central axis, a rear housing provided at a distal end of the housing, wherein the distal end is pointing away from a medicament delivery site during use of the medicament delivery device, and a plunger rod rotatable between a first rotational position and a second rotational position, wherein the plunger rod is axially fixed with respect to the rear housing in the first rotational position and axially moveable with respect to the rear housing in the second rotational position. A medicament delivery member cover is coupled to the housing and movable between an extended position and a retracted position, wherein the rear housing comprises a rear housing boss configured to engage with a plunger rod profile to block axial movement of the plunger rod with respect to the rear housing in the first rotational position, wherein the medicament delivery member cover comprises a stop rib configured to engage with an outer boss of the plunger rod in the extended position, wherein the engagement is configured to block a movement from of the plunger rod from the first rotational position to the second rotational position, wherein, when the medicament delivery member cover is pushed into the retracted position, the stop rib disengages the outer boss of the plunger rod and the plunger rod is rotated into the second rotational position, wherein the plunger rod profile disengages the rear housing boss in the second rotational position.

### TECHNICAL FIELD

The present disclosure relates to a medicament delivery device and in particular to a medicament delivery device comprising a rotatable plunger rod.

### BACKGROUND

A medicament delivery device, in particular an autoinjector, according to the prior art comprises a housing comprising a front housing and a rear housing. The housing is adapted to hold a medicament container, such as a syringe. The syringe may be a pre-filled syringe and have a needle as a medicament delivery member arranged at a proximal end. When the autoinjector and/or the syringe are assembled, a protective needle sheath may be removably coupled to the needle. The protective needle sheath may be made of a flexible shell (for instance composed of rubber) and/or a rigid shell (for instance composed of plastic). A stopper, also called plunger, may be arranged for sealing the syringe distally and for expelling a medicament contained in the syringe through the needle. In other exemplary embodiments, the medicament container may be a cartridge which includes the medicament and engages a removable needle (e.g., by threads, snaps, friction, etc.).

A cap may be removably disposed at a proximal end of the housing. The cap may include an element (e.g., a barb, a hook, a narrowed section, etc.) arranged to engage the protective needle sheath, the housing and/or a medicament delivery member cover, also referred to as needle cover, telescoped within the housing. The cap may comprise grip features for facilitating removal of the cap (e.g., by twisting and/or pulling the cap relative to the housing).

A medicament delivery member cover biasing member may be arranged to bias the medicament delivery member cover in a proximal direction relative to the housing, before injection, for safety reasons. The medicament delivery member cover biasing member needs to be compressed at the beginning of injection in order to retract the medicament delivery member cover inside the housing and uncover the medicament delivery member, thereby activating the device.

A pre-loaded drive biasing member, for instance a pre-compressed drive biasing member, may be arranged within the housing. A plunger rod may be used to forward force from the drive biasing member to the stopper. In an exemplary embodiment, the plunger rod is hollow and the drive biasing member is arranged within the plunger rod. In another exemplary embodiment, the plunger rod is solid and the drive biasing member engages a distal end of the plunger rod. When the pre-loaded drive biasing member is released, the drive biasing member biases the plunger rod in the proximal direction relative to the housing, releasing the plunger rod. Besides that, the plunger rod may itself comprise means for sealing the medicament container such that no stopper needs to be provided.

A plunger rod release mechanism may be arranged for preventing release of the plunger rod prior to retraction of the medicament delivery member cover relative to the housing and for releasing the plunger rod only once the medicament delivery member cover is sufficiently retracted.

A medicament delivery member cover lock mechanism (needle cover lock mechanism) may be arranged to prevent retraction of the medicament delivery member cover relative to the housing when the cap is in place, thereby avoiding unintentional activation of the autoinjector (e.g., if dropped, during shipping or packaging, etc.). Such mechanisms are known in the art and will not be described in detail.

However, there is a need to further simplify the structure of medicament delivery devices while effectively preventing inadvertent activation of the device.

This need is met by a device according to the present disclosure.

### SUMMARY

The present disclosure relates to a medicament delivery device comprising a housing having a longitudinal central axis, a rear housing provided at a distal end of the housing, wherein the distal end is pointing away from a medicament delivery site during use of the medicament delivery device, and a plunger rod rotatable between a first rotational position and a second rotational position, wherein the plunger rod is axially fixed with respect to the rear housing in the first rotational position and axially moveable with respect to the rear housing in the second rotational position. A pre-loaded drive biasing member exerts a force on the plunger rod in the proximal direction. A medicament delivery member cover is coupled to the housing and movable between an extended position and a retracted position, wherein the rear housing comprises a rear housing boss configured to engage with a plunger rod profile to block axial movement of the plunger rod with respect to the rear housing in the first rotational position, wherein the medicament delivery member cover comprises a stop rib configured to engage with an outer boss of the plunger rod in the extended position, wherein the engagement is configured to block a movement from of the plunger rod from the first rotational position to the second rotational position, wherein, when the medicament delivery member cover is pushed into the retracted position, the stop rib disengages the outer boss of the plunger rod and the plunger rod is rotated into the second rotational position, wherein the plunger rod profile disengages the rear housing boss in the second rotational position.

Various embodiments may preferably implement the following features.

Preferably, the plunger rod profile is sloped and configured to guide the rotation of the plunger rod from the first rotational position to the second rotational position.

Preferably, the plunger rod profile extends in a circumferential direction of the plunger rod.

Preferably, the plunger rod profile protrudes from the plunger rod in a radial direction.

Preferably, the plunger rod profile is formed as a recess.

Preferably, the plunger rod profile is formed as a cut-out.

Preferably, the plunger rod comprises a substantially hollow cylindrical part at the distal end of the plunger rod having a bigger radius than the remaining plunger rod, wherein the plunger rod profile and the outer boss are located on the substantially cylindrical part.

The present disclosure further relates to a medicament delivery device comprising a housing having a longitudinal central axis, a rear housing provided at a distal end of the housing, wherein the distal end is pointing away from a medicament delivery site, and a plunger rod rotatable between a first rotational position and a second rotational position, wherein the plunger rod is axially fixed with respect to the rear housing in the first rotational position and axially moveable with respect to the rear housing in the second rotational position, a pre-loaded drive biasing member exerting a force on the plunger rod in the proximal direction, a medicament delivery member cover coupled to the housing and movable between an extended position and a retracted position, wherein the rear housing comprises an inner part and the plunger rod comprises a hollow cylindrical part at the distal end of the plunger rod, wherein the inner part is positioned inside the hollow cylindrical part in a radial direction with respect to the central axis, the inner part comprising a rear housing profile configured to engage with an inner boss of the plunger rod to block axial movement of the plunger rod with respect to the rear housing in the first rotational position, wherein the medicament delivery member cover comprises a stop rib configured to engage with an outer boss of the plunger rod in the extended position, wherein the engagement is configured to block a movement of the plunger rod from the first rotational position to the second rotational position, wherein, when the medicament delivery member cover is pushed distally into the retracted position, the stop rib disengages the outer boss of the plunger rod and the plunger rod is rotated into the second rotational position, wherein the inner boss disengages the rear housing profile in the second rotational position.

Various embodiments may preferably implement the following features.

Preferably, the rear housing profile is sloped and configured to aid the rotation of the plunger rod from the first rotational position to the second rotational position.

Preferably, the inner part is substantially formed as a hollow cylinder.

Preferably, the rear housing profile is formed as a recess on an outer circumferential surface of the inner part.

Preferably, the rear housing profile is formed as a cut-out on an outer circumferential surface of the inner part.

Preferably, the rear housing profile extends in a circumferential direction on an outer surface of the inner part and has an opening in a proximal direction towards the medicament delivery site.

The invention is specified by the independent claims. Preferred embodiments are defined in the dependent claims. In the following description, although numerous features may be designated as optional, it is nevertheless acknowledged that all features comprised in the independent claims are not to be read as optional.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device.

When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a longitudinal axis extending in the direction of the longest extension of the device and/or component. Similarly, “radial” or “radially” refer to a direction extending radially relative to the axis, and “rotation”, “rotational” and “rotationally” refer to rotation relative to the axis.

When a component is said to move proximally, distally, axially in a proximal direction, axially in a distal direction or equivalent terms, the movement is relative to the housing of the injection device, unless mentioned otherwise.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1A shows a partial view of a plunger rod according to an example.

FIG. 1B shows a partial view of a rear housing according to an example.

FIG. 1C shows a partial view of a medicament delivery member cover according to an example.

FIG. 2 shows a partial view of a medicament delivery device according to an example in an assembled state.

FIG. 3 shows a partial view of a medicament delivery device according to an example in an activation state.

FIG. 4 shows an exploded view of a medicament delivery device according to the example of FIGS. 1 to 3.

FIG. 5A shows a partial view of a plunger rod according to an example.

FIG. 5B shows a perspective view of the plunger rod according to FIG. 5A.

FIG. 5C shows a rear housing according to an example.

FIG. 5D shows a partial view of a medicament delivery member cover according to an example.

FIGS. 6A and 6B show partial views of a medicament delivery device according to an example in an assembled state.

FIGS. 7A and 7B show partial views of a medicament delivery device according to an example in an activation state.

FIG. 8 shows an exploded view of a medicament delivery device according to the example of FIGS. 5 to 7.

### DETAILED DESCRIPTION

The activation mechanism according to the present invention can be employed for various medicament delivery devices. Thus, only the parts relevant for carrying out the disclosure will be described in detail.

In the figures, elements not essential for carrying out the invention have been omitted. In particular, not all figures show a complete outer housing, biasing members, a medicament container, cap, etc. in order not to render the depictions overly complicated and not to blur the scope of the disclosure.

In the following, the assembled state refers to a state in which the device is delivered to the patient, i.e., before activation and medicament delivery. In the assembled state, the plunger rod is in a locked position (first rotational position). In the activation state, the plunger rod is free to rotate, but still substantially locked in the axial direction. In the activated state (second rotational position), the plunger rod is free to move in the proximal direction for medicament delivery. The plunger rod may thus be displaceable along the axial direction.

The medicament delivery member cover can be moved from an extended to a retracted position. Therefore, the medicament delivery member cover can be displaced in an axial direction. In the extended position, the medicament delivery member cover extends in a proximal direction from the housing. Thereby, a medicament delivery member, e.g., a needle, may be covered by the medicament delivery member cover. By pushing the medicament delivery member cover in the distal direction, e.g., by pushing the device onto a medicament delivery site, the medicament delivery member cover is moved to the retracted position for activating medicament delivery. After delivery, in particular after removal of the device from a patient's skin, the medicament delivery member cover may be moved back to the extended position to cover the medicament delivery member and prevent injuries of the patient. In some embodiments, the medicament delivery member cover may also extend further than the extended position in a locked position.

In the following, the term engagement encompasses any kind of interaction between the named features such as abutment, clamping, locking etc.

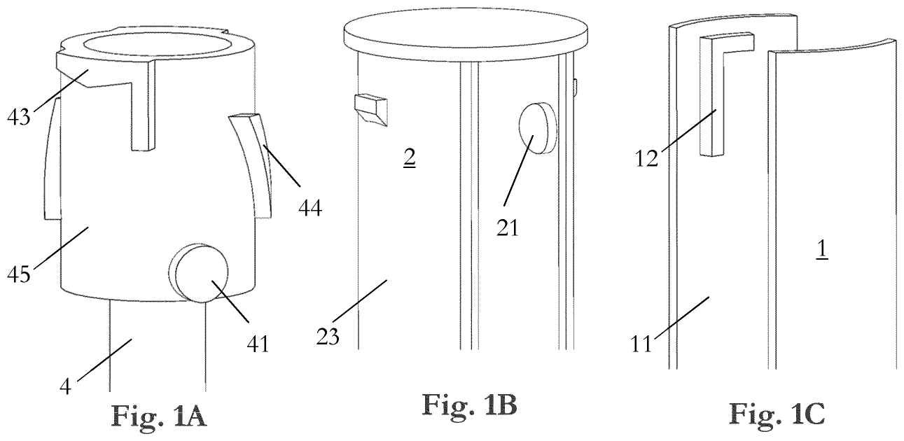

FIG. 1A shows a plunger rod 4 according to an example of the present disclosure. The plunger rod 4 comprises an outer boss 41 (outer plunger rod knob) and a plunger rod profile 43. Moreover, the plunger rod 4 may comprise a plunger rod sloped profile 44 and a substantially cylindrical part 45. The proximal part of the plunger rod 4 may be provided with a stopper or the like as described above, and a drive biasing member may act on the distal part of the plunger rod 4 to provide a force in the proximal direction and expel a medicament after activation of the medicament delivery device. The plunger rod 4 may comprise a substantially cylindrical part 45 at the distal end of the plunger rod 4, wherein the cylindrical part 45 may be hollow and wider than the proximal part of the plunger rod 4 in a radial direction (i.e., having a larger radius). The drive biasing member may extend inside the plunger rod 4 and act on an inner proximal end surface of the plunger rod 4. The drive biasing member may thus extend almost the whole length of the plunger rod 4. Alternatively, the drive biasing member may act on the hollow part of the cylindrical part 45. However, the cylindrical part 45 may also be solid (not shown in the figures) and the drive biasing member may thus act on the distal end of the cylindrical part.

The plunger rod sloped profile 44 may be used during assembly to guide the plunger rod 4 into its initial position (assembled position).

The outer boss 41, the plunger rod profile 43 and the plunger rod sloped profile 44 may be located on an outer circumferential surface of the cylindrical part 45.

The plunger rod profile 43 may extend in a circumferential direction of the cylindrical part 45. The plunger rod profile 43 may substantially have an L-shape, wherein the profile 43 extends in the circumferential direction and the proximal direction of the cylindrical part 45. In the figures, the plunger rod profile 43 is located at the distal end of the cylindrical part 45. However, the plunger rod profile 43 may also be provided further proximally. The interacting means to be described below might then have to be adapted accordingly.

FIG. 1B shows a partial view of a rear housing 2 according to an example which may also be referred to as rear cap. The rear housing 2 comprises at least one rear housing boss 21. The rear housing 2 may further comprise two or more rear housing arms 23 extending in the proximal direction. The rear housing boss 21 may be provided on each of the rear housing arms 23 on an inner side in a radial direction. However, the rear housing 2 may also be formed as a (hollow) cylindrical body. Thus, the rear housing boss 21 may be provided on an inner circumferential surface of the cylindrical body.

FIG. 1C shows a partial view of a medicament delivery member cover 1 (needle cover) according to an example. The medicament delivery member cover 1 comprises at least one stop rib 12. The medicament delivery member cover 1 may comprise two or more arms 11 extending in the distal direction. A stop rib 12 may be provided on a distal end of each of the medicament delivery member cover arms 11.

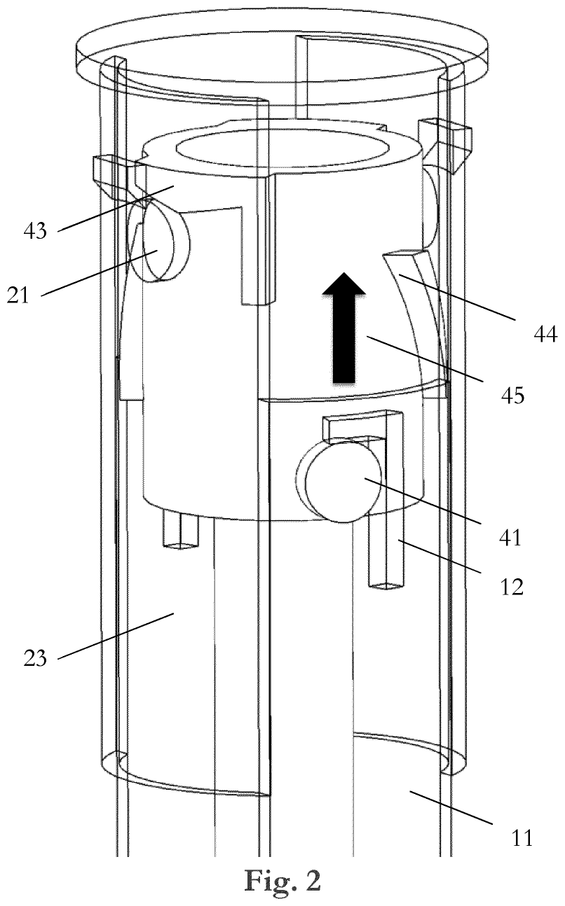

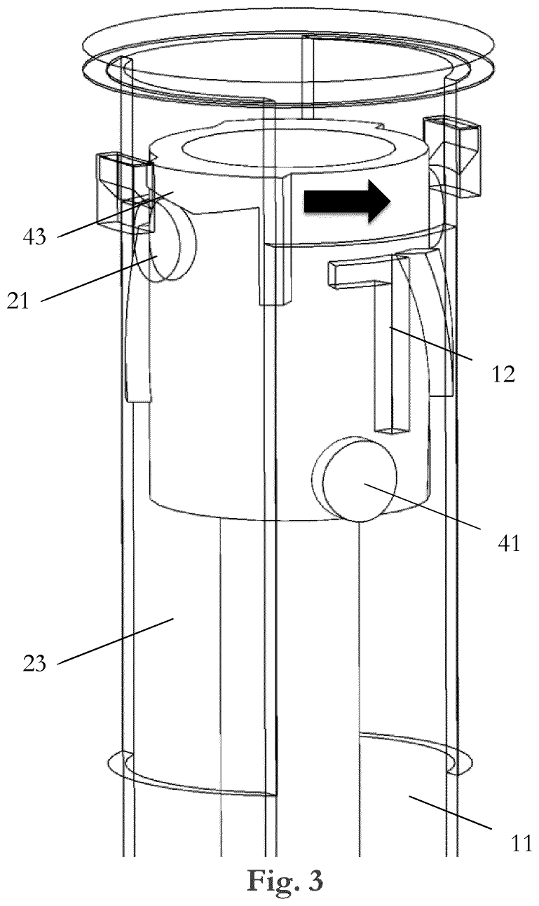

FIG. 2 shows a partial view of the distal end of the device in an assembled state, wherein the plunger rod 4 is in the first rotational position. Biasing member(s), a housing 3 (visible in FIG. 3), a medicament container etc. have been omitted in this view and both the rear housing 2 as well as the medicament delivery member cover 1 are transparently shown. The two arms 23 of the rear housing 2 and the two arms 11 of the medicament delivery member cover 1 are offset by 90 degrees in an alternating order. The medicament delivery member cover 1 is in its extended position.

As can be seen from FIG. 2, the outer boss 41 of the plunger rod 4 abuts the stop rib 12 of the medicament delivery member cover 1 in the circumferential direction and the plunger rod profile 43 abuts the rear housing boss 21 in the axial direction. In this first rotational position, the engagement between the plunger rod profile 43 and the rear housing boss 21 prevents a movement of the plunger rod 4 in the proximal direction, while in the shown example, the plunger rod profile 43 is sloped in order to initiate or aid a rotational movement of the plunger rod 4 (rightwards in FIG. 2). The abutment of the outer boss 41 with the stop rib 12 prevents a rotational movement of the plunger rod 4. In other words, the plunger rod 4 is fixed in axial and rotational direction.

As indicated by the arrow in FIG. 2, the medicament delivery member cover 1 is pushed in the distal direction from the extended position to the retracted position for activating the device, e.g., by pushing the device onto a medicament delivery site.

FIG. 3 shows the device of FIG. 2 in the activation state, wherein the medicament delivery member cover 1 is in the retracted position. In this position, the stop rib 12 disengages the outer boss 41 of the plunger rod 4 and the plunger rod 4 can be rotated into the second rotational position (see arrow). The rotational movement of the plunger rod 4 may be guided by a slope in the plunger rod profile 43 as shown in the figures. The plunger rod profile 43 may thus be configured to aid rotation of the plunger rod 4. After a predetermined rotation, the plunger rod 4 reaches the second rotational position (not shown in the figures). In the second rotational position, the plunger rod profile 43 disengages the rear housing boss 21 and the plunger rod 4 is free to be moved in the proximal direction by the drive biasing member to expel the medicament.

The plunger rod profile 43 may extend in a circumferential direction of the plunger rod 4 and may protrude from the plunger rod 4 in a radial direction.

The plunger rod profile may alternatively be formed as a recess or a cut-out. In the latter two cases, the profiles are inverse compared to the protruding profile.

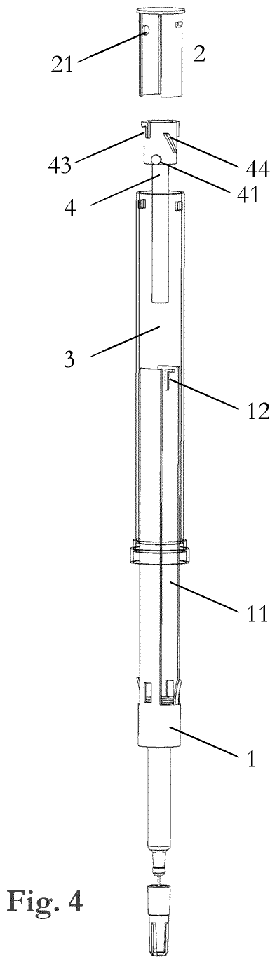

FIG. 4 shows an exploded view of the device as shown in FIGS. 1 to 3.

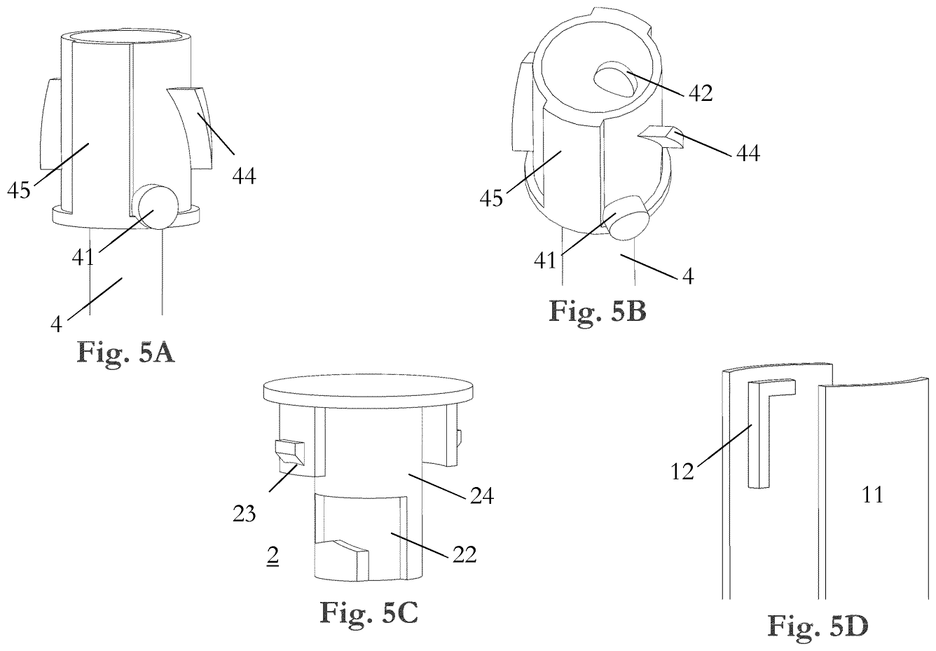

FIGS. 5 to 8 show a further example of the present disclosure which is related to the above-described example. Therefore, only the elements and functions differing from the above example will be described in detail.

FIG. 5A shows a plunger rod 4 according to an example of the present disclosure. The plunger rod 4 comprises an outer boss 41 (outer plunger rod knob) and may comprise a plunger rod sloped profile 44 and a substantially cylindrical part 45. The outer boss 41 is provided at a proximal end of the cylindrical part 45 according to this example, but may also be moved further distally. Other elements might have to be adapted accordingly. The proximal part of the plunger rod 4 may be provided with a stopper or the like as described above, and a drive biasing member may act on the distal part of the plunger rod 4 to provide a force in the proximal direction and expel a medicament after activation of the medicament delivery device (see above). The plunger rod 4 may comprise a substantially cylindrical part 45 at the distal end of the plunger rod 4, wherein the cylindrical part 45 may be hollow and wider than the proximal part of the plunger rod 4 in a radial direction (i.e., having a larger radius).

The plunger rod sloped profile 44 may be used during assembly to guide the plunger rod 4 into its initial position (assembled position).

The outer boss 41, and the plunger rod sloped profile 44 may be located on an outer circumferential surface of the cylindrical part 45.

As shown in FIG. 5B, the plunger rod 4 further comprises an inner boss 42 (inner plunger rod knob) located in an inner circumferential surface of the hollow cylindrical part 45.

FIG. 5C shows a partial view of a rear housing 2 according to an example which may also be referred to as rear cap. The rear housing 2 comprises at least one rear housing profile 22. The rear housing 2 may further comprise two or more rear housing arms 23 extending in the proximal direction. The rear housing profile 22 may be provided on a rear housing inner part 24, particularly on an outer circumferential surface thereof.

The rear housing inner part 24 may be provided as a substantially cylindrical element extending in a proximal direction from the distal end of the rear housing 2. The outer diameter of the rear housing inner part 24 may be smaller than the inner diameter of the hollow cylindrical part 45 such that the rear housing inner part 24 can be positioned in the distal end of the plunger rod 4, i.e., the hollow cylindrical part 45.

The rear housing profile 22 may be provided as a recess or cut-out. The rear housing profile 22 may extend in a circumferential direction of the rear housing inner part 24 and have an opening in a proximal direction such that the plunger rod 4 can be moved in the proximal direction in the second rotational position. That is, the rear housing profile may substantially have an L-shape.

FIG. 5D shows a partial view of a medicament delivery member cover 1 (needle cover) according to an example. The medicament delivery member cover 1 substantially corresponds to the one according to FIG. 1C.

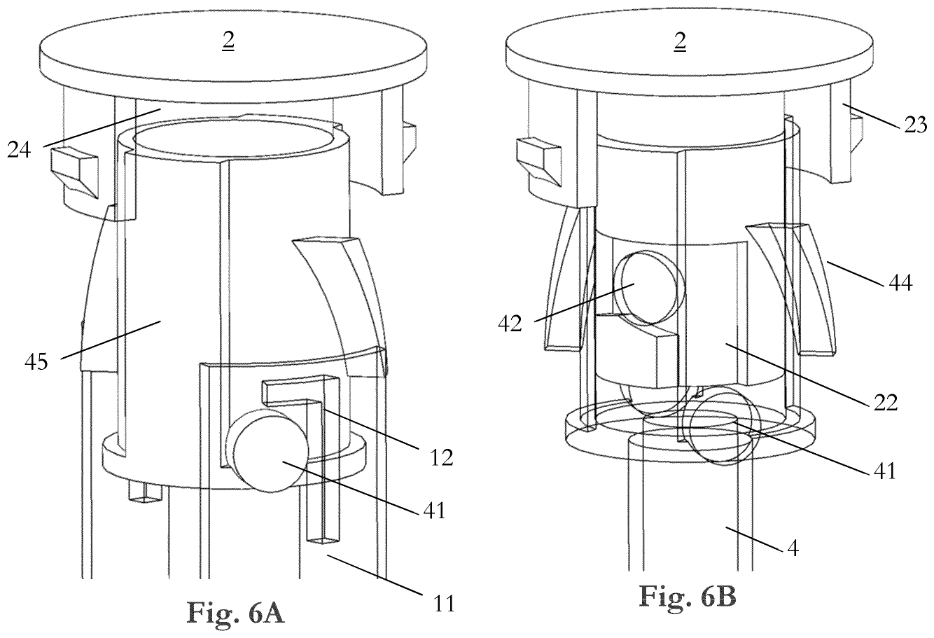

FIGS. 6A and 6B are depictions of the device according to an example in the assembled state. In FIG. 6A, the medicament delivery member cover 1 is transparently displayed and in FIG. 6B, the medicament delivery member cover is omitted while the plunger rod 4 is transparently displayed. The plunger rod 4 is in its first rotational position and the medicament delivery member cover 1 is in its extended position.

As described above, the outer boss 41 abuts the stop rib 12, thus preventing rotational movement of the plunger rod 4 (FIG. 6A). Moreover, the inner boss 42 engages the rear housing profile 22, thus blocking axial movement in the proximal direction (FIG. 6B).

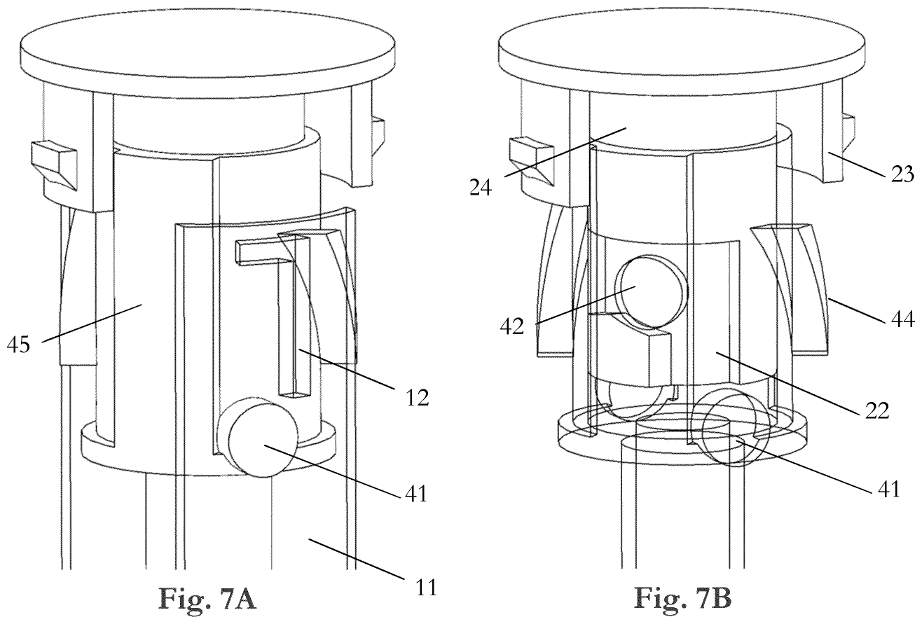

FIGS. 7A and 7B show the device of FIGS. 6A and 6B in the activation state. That is, the medicament delivery member cover 1 is in its retracted position and the plunger rod 4 still in the first rotational position.

When the medicament delivery member cover 1 is pushed distally into the retracted position, e.g., by pushing the device onto a medicament delivery site, the stop rib 12 disengages the outer boss 41 of the plunger rod 4 as shown in FIG. 7A. The plunger rod 4 is free to rotate into the second rotational position (not shown). The rotational movement may be guided or aided by a slope in the rear housing inner profile 22 (FIG. 7B). After a certain amount of rotation, the plunger rod 4 has reached the second rotational position in which the inner boss 42 disengages the rear housing profile 22. The plunger rod 4 is thus free to be moved in the proximal direction to expel the medicament.

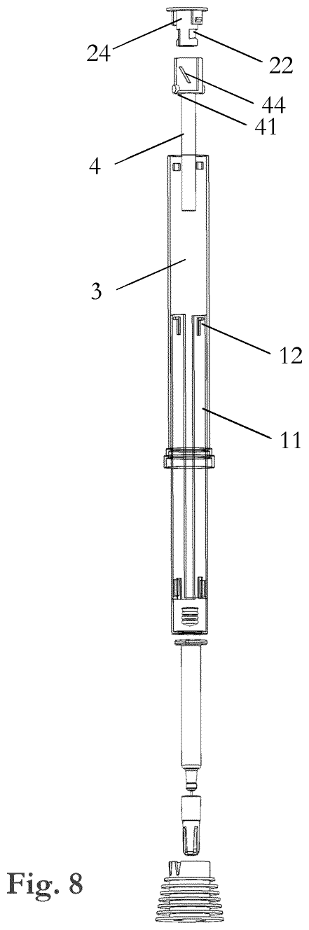

FIG. 8 shows an exploded view of the device as described in FIGS. 5 to 7.

According to the present disclosure, the plunger rod 4 is in tension via a drive biasing member (“powerpack”) and the interaction of bosses and profiles described above hold it in place. Only when the interaction is released by pushing the medicament delivery member cover 1 in the distal direction, thereby activating the device, the plunger rod 4 can rotate out of the engagement to be moved in a proximal direction for medicament delivery.

At the end of the plunger rod profile 43 or the rear housing profile 22 when viewed in a rotational direction, a protrusion or step may be provided which protrudes in the distal direction and which has to be overcome by the plunger rod 4 before release. This might add a further security to prevent inadvertent rotation and slightly compress the drive biasing member further prior to medicament delivery such that dose delivery may be improved.

By providing a plunger rod having a profile and thus not requiring a profile on the housing as disclosed herein, the complexity of manufacturing the housing can be reduced. Moreover, the size of the plunger rod can be reduced such that it might entirely fit into a syringe at the end of the dose given.

Moreover, by providing a housing part inside the plunger rod, the constructional rigidity of the device can be increased. Also, this provides further design freedom, particularly for the interaction with the medicament delivery member cover/needle cover, since the profile interaction with the housing resides inside the plunger rod.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDw123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

1-13. (canceled)

14. A medicament delivery device, comprising:
a housing having a longitudinal central axis;
a rear housing provided at a distal end of the housing, wherein the distal end is pointing away from a medicament delivery site during use of the medicament delivery device;
a plunger rod rotatable between a first rotational position and a second rotational position, wherein the plunger rod is axially fixed with respect to the rear housing in the first rotational position and axially moveable with respect to the rear housing in the second rotational position;
a pre-loaded drive biasing member exerting a force on the plunger rod in the proximal direction; and
a medicament delivery member cover coupled to the housing and movable between an extended position and a retracted position,
wherein the rear housing comprises a rear housing boss configured to engage with a plunger rod profile to block axial movement of the plunger rod with respect to the rear housing in the first rotational position,
wherein the medicament delivery member cover comprises a stop rib configured to engage with an outer boss of the plunger rod in the extended position, wherein the engagement is configured to block a movement from of the plunger rod from the first rotational position to the second rotational position,
wherein, when the medicament delivery member cover is pushed into the retracted position, the stop rib disengages the outer boss of the plunger rod and the plunger rod is rotated into the second rotational position, wherein the plunger rod profile disengages the rear housing boss in the second rotational position.

15. The medicament delivery device according to claim 14, wherein the plunger rod profile is sloped and configured to guide the rotation of the plunger rod from the first rotational position to the second rotational position.

16. The medicament delivery device according to claim 14, wherein the plunger rod profile extends in a circumferential direction of the plunger rod.

17. The medicament delivery device according to claim 14, wherein the plunger rod profile protrudes from the plunger rod in a radial direction.

18. The medicament delivery device according to claim 14, wherein the plunger rod profile is formed as a recess.

19. The medicament delivery device according to claim 14, wherein the plunger rod profile is formed as a cut-out.

20. The medicament delivery device according to claim 14, wherein the plunger rod comprises a substantially hollow cylindrical part at the distal end of the plunger rod having a bigger radius than the remaining plunger rod, wherein the plunger rod profile and the outer boss are located on the substantially cylindrical part.

21. A medicament delivery device, comprising:
a housing having a longitudinal central axis;
a rear housing provided at a distal end of the housing, wherein the distal end is pointing away from a medicament delivery site;
a plunger rod rotatable between a first rotational position and a second rotational position, wherein the plunger rod is axially fixed with respect to the rear housing in the first rotational position and axially moveable with respect to the rear housing in the second rotational position;
a pre-loaded drive biasing member exerting a force on the plunger rod in the proximal direction; and
a medicament delivery member cover coupled to the housing and movable between an extended position and a retracted position,
wherein the rear housing comprises an inner part and the plunger rod comprises a hollow cylindrical part at the distal end of the plunger rod,
wherein the inner part is positioned inside the hollow cylindrical part in a radial direction with respect to the central axis,
the inner part comprising a rear housing profile configured to engage with an inner boss of the plunger rod to block axial movement of the plunger rod with respect to the rear housing in the first rotational position,
wherein the medicament delivery member cover comprises a stop rib configured to engage with an outer boss of the plunger rod in the extended position, wherein the engagement is configured to block a movement of the plunger rod from the first rotational position to the second rotational position,
wherein, when the medicament delivery member cover is pushed distally into the retracted position, the stop rib disengages the outer boss of the plunger rod and the plunger rod is rotated into the second rotational position, wherein the inner boss disengages the rear housing profile in the second rotational position.

22. The medicament delivery device according to claim 21, wherein the rear housing profile is sloped and configured to aid the rotation of the plunger rod (4) from the first rotational position to the second rotational position.

23. The medicament delivery device according to claim 21, wherein the inner part is substantially formed as a hollow cylinder.

24. The medicament delivery device according to claim 21, wherein the rear housing profile is formed as a recess on an outer circumferential surface of the inner part.

25. The medicament delivery device according to claim 21, wherein the rear housing profile is formed as a cut-out on an outer circumferential surface of the inner part.

26. The medicament delivery device according to claim 21, wherein the rear housing profile extends in a circumferential direction on an outer surface of the inner part and has an opening in a proximal direction towards the medicament delivery site.
